(19) United States
(12) Patent Application Publication
(10) Pub. No.: US 2016/0228482 A1
(43) Pub. Date: Aug. 11, 2016

(54) TEA TREE OIL DELIVERY SYSTEM

(71) Applicant: Zena Pharm Pty Ltd, Nicholasville, KY (US)

(72) Inventors: Jeremy Delk, Nicholasville, KY (US); Mark Linford, Bendigo (AU)

(21) Appl. No.: 14/705,393

(22) Filed: May 6, 2015

Related U.S. Application Data
(60) Provisional application No. 61/989,042, filed on May 6, 2014.

(51) Int. Cl. A61K 36/61; A61K 9/00; A61K 47/44; A61K 47/02; A61K 47/10

(52) U.S. Cl. CPC A61K 36/61; A61K 47/02; A61K 47/10; A61K 47/44; A61K 9/0014

(57) ABSTRACT

The present invention provides a novel composition that functions as a carrier effective for administering tea tree oil to alleviate the conditions of the skin, such as basal cell carcinoma and actinic keratosis.

### RELATED APPLICATIONS

This application claims priority to U.S. Provisional Application 61/989,042, filed May 6, 2014, which is hereby incorporated by reference in its entirety.

### FIELD OF THE INVENTION

The invention relates to a novel delivery system for transdermal delivery of compounds. The delivery system can be utilized particularly to treat conditions of the skin and can be beneficial in successfully delivering compounds to subdermal sites.

### BACKGROUND

Topical application of compounds is desirable benefit for site specific delivery to alleviate difficulties with the skin of a subject. However, the skin is a difficult organ for site specific delivery as it is composed of many layers and designed as a barrier for both retention of matter within the subject and for preventing entry of matter into the subject. Compounds aimed at targeting a condition within the skin or subdermally thereto often need the assistance of a carrier to properly penetrate the skin and reach the area where action is required. The chemistry of the carrier allows for passage across the epidermal and dermal layers of the attached compound. The present invention provides a novel carrier that is potent in delivering agents transdermally.

### SUMMARY OF THE INVENTION

The present invention provides a pharmaceutical composition, comprising a therapeutically effective amount of a therapeutic agent for the skin, such as tea tree oil (Melaluca altemifolia), and a pharmaceutically acceptable carrier. The pharmaceutical composition may include between about 9.2 and about 10.8 weight percent tea tree oil and between about 87.95 and about 90.5 weight percent pharmaceutically acceptable carrier. In other embodiments, the pharmaceutical composition may include about 10 weight percent tea tree oil and about 90 weight percent carrier.

The pharmaceutically acceptable carrier may include water, propylene glycol, phenoxyethanol, heavy paraffin oil, soft white paraffin, cetostearyl alcohol and macrogol cetrostearyl ether. In some embodiments, the carrier may comprise between about 46.5 and about 49.5 weight percent water, about 4.2 and about 5.8 weight percent propylene glycol, about 0.4 and about 0.8 weight percent phenoxyethanol, about 9.2 and about 10.8 weight percent heavy paraffin oil, about 9.2 and about 10.8 weight percent soft white paraffin, about 14.2 and about 15.6 weight percent cetostearyl alcohol, and about 0.4 and about 1.0 weight percent macrogol cetostearyl ether. In other embodiments, the carrier may comprise about 47.9 weight percent water, 5.0 weight percent propylene glycol, 0.6 weight percent phenoxyethanol, 10.0 weight percent heavy paraffin oil, 10.0 weight percent soft white paraffin, 14.8 weight percent cetostearyl alcohol and 0.7 weight percent macrogol cetostearyl ether.

The pharmaceutical composition may include vanillin. In some embodiments, between about 0.75 and about 1.25 weight percent vanilln. In other embodiments, the composition may comprise about 1.0 weight percent vanillin.

The present invention also provides methods of treating skin conditions, such as actinic keratosis and basal cell carcinoma, comprising topically applying to a subject in need thereof the pharmaceutical compositions of the invention.

### BRIEF DESCRIPTION OF THE DRAWINGS

FIG. 1 shows that tumor treatment with tea tree oil and the carrier does not cause dermal inflammation by skin inflammation score.

FIG. 2 shows that inflammatory cell infliltraion is similar to untreated skin as compared to treated with DMSO.

FIG. 3 shows that the applied tea tree oil with the carrier inhibits B16 melanoma growth in the murine model.

FIG. 4 shows a comparison of the subsequent tumor weights in treated and untreated subjects.

FIG. 5 shows three independent studies of the effect of tea tree oil and the carrier on tumor volume.

FIG. 6 shows a comparison between treatment and non-treatment applied on day three after tumor implantation.

FIG. 7 shows a comparison of subcutaneous tumor size between treated and non-treated subjects.

FIG. 8 shows mean tumor weight between treated and untreated subjects.

### DESCRIPTION

Pharmaceutical compositions are provided for the treatment of actinic keratosis (AK), melanoma or basal cell carcinoma. In one embodiment the pharmaceutical composition comprises a therapeutically effective amount of tea tree oil (Melaluca altemifolia) and a pharmaceutically effective carrier. In one possible embodiment the pharmaceutical composition further includes vanillin.

In one possible embodiment, the pharmaceutically acceptable carrier includes water, propylene glycol, phenoxyethanol, heavy paraffin oil, soft white paraffin, cetostearyl alcohol and macrogol cetrostearyl ether.

In one possible embodiment, the pharmaceutical composition includes between about 9.2 and about 10.8 weight percent tea tree oil and between about 87.95 and about 90.05 weight percent pharmaceutically acceptable carrier. That embodiment may also include between about 0.75 and about 1.25 weight percent vanillin.

In one embodiment the pharmaceutically acceptable carrier includes between about 46.5 and about 49.5 weight percent water, about 4.2 and about 5.8 weight percent propylene glycol, about 0.4 and about 0.8 weight percent phenoxyethanol, about 9.2 and about 10.8 weight percent heavy paraffin oil, about 9.2 and about 10.8 weight percent soft white paraffin, about 14.2 and about 15.6 weight percent cetostearyl alcohol, and about 0.4 and about 1.0 weight percent macrogol cetostearyl ether.

In one embodiment the pharmaceutically acceptable carrier includes about 47.9 weight percent water, 5.0 weight percent propylene glycol, 0.6 weight percent phenoxyethanol, 10.0 weight percent heavy paraffin oil, 10.0 weight percent soft white paraffin, 14.8 weight percent cetostearyl alcohol and 0.7 weight percent macrogol cetostearyl ether.

Those skilled in the art will appreciate that the therapeutic agent can be any known therapeutic, such as a hormone, small molecule, peptide, nucleotide, vitamin, vaccine, antigen, antibody or fragment thereof, or organic chemical. Those skilled in the art will appreciate that the therapeutic agent can be applied with the carrier to a subject at any point of a subject's skin. Those skilled in the art will appreciate that the composition can be applied at a particular site, such as a site in need of treatment. Those skilled in the art will further appreciate that application of the composition at any point can further provide systemic administration, such that the agent can be delivered to a site in need thereof that is distal to the point of application. Those skilled in the art will further appreciate that the therapeutic agent can comprise more than one agent. For example, the therapeutic agent may comprise an anti-cancer agent, such as a skin cancer agent (e.g. tea tree oil, fluorouracil, imiquimod, visodegib, aldesleukin, dabrafenib, dacarbazine, recombinant interferon α-2b, ipilimumab, pembrolizumab, trametinib, nivulomab, and vemurafenib).

A method of treating AK comprises topically applying to a subject in need a pharmaceutically effective amount of the pharmaceutical composition. Between about 0.5 and about 1 gram of the pharmaceutical composition is applied per 10 cm² of skin surface area between 2 and 3 times a day.

The pharmaceutical composition is prepared by combining/mixing appropriate amounts of water, propylene glycol and phenoxyethanol together and heating to between about 70-75° C. Appropriate amounts of heavy paraffin oil, soft white paraffin, cetostearyl alcohol and macrogel cetrostearyl ether are blended together in another vessel and heated to between about 75-80° C. All of the ingredients are then added together and mixed for about one minute or until homogenized.

The homogenized mixture is then cooled to about 40-45° C. and an appropriate amount of vanillin is then added with further mixing.

Afterwards, the mixture is allowed to stand for 48 hours at room temperature. An appropriate amount of tea tree oil is then added with continual mixing to homogenize the pharmaceutical composition.

### EXAMPLES

Tea tree oil can be effective in treating precancerous actinic keratosis (see, e.g., Demelza et al., Journal of Dermatological Science, Volume 67, Issue 2, August 2012, Pages 120-129). This study demonstrated that Tea Tree Oil (TTO) can significantly reduce the viability in vitro of 2 murine tumor cell lines: AE17 and B16 in a dose and time dependent manner. The in vivo part of the study showed that 3% and 10% TTO can inhibit tumor growth in mice. 10% TTO in DMSO was able to cause regression of tumors. DMSO can enhance penetration of substances through skin (transdermal enchancer). Unfortunately treatment with 10% TTO/DMSO was limited to 4 days limited to 4 days due to development of severe irritation, allowing regrowth of the tumors. The following example demonstrates that the carrier of the invention can deliver TTO without the irritation seen in other carriers such as DMSO.

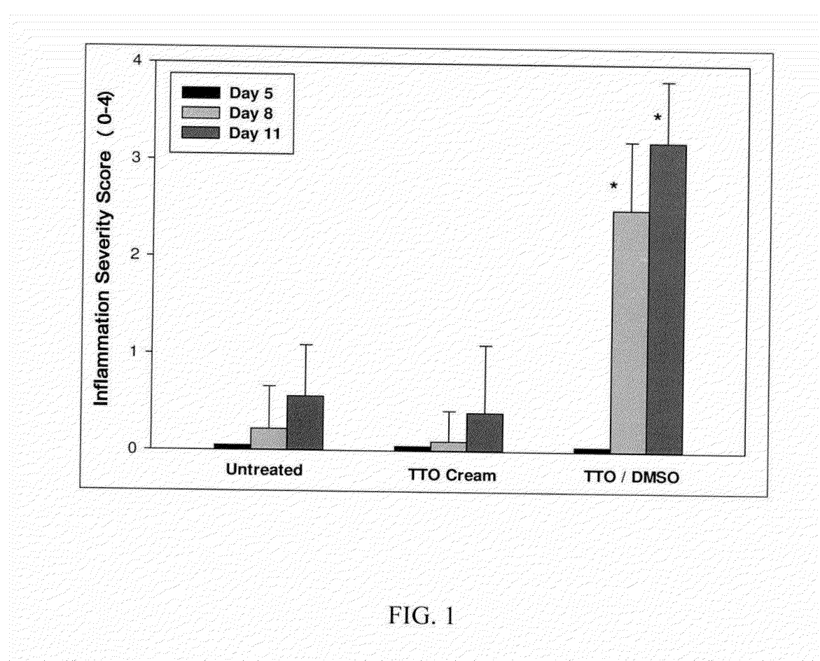

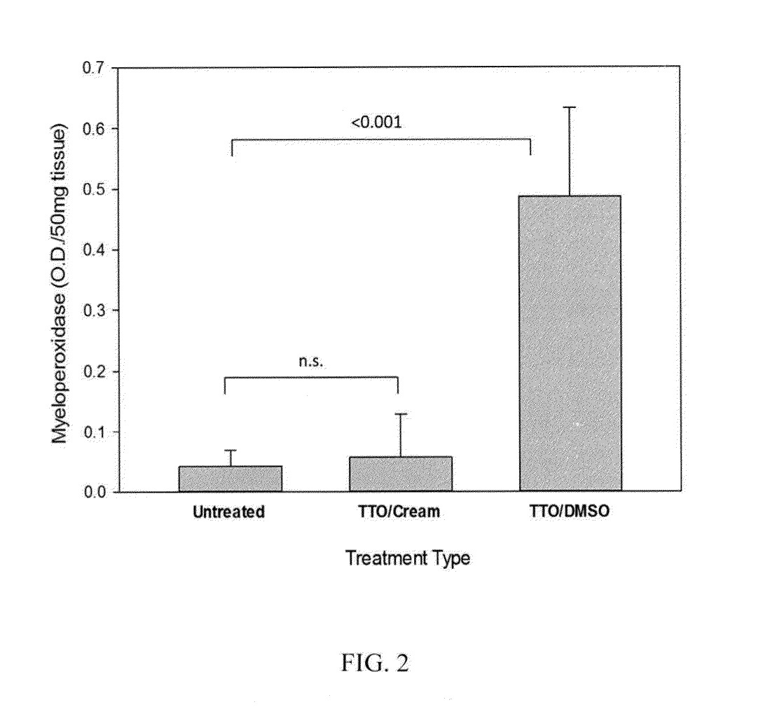

To test the carrier, tumors were implanted into murine models and allowed to grow for before treatment was implemented. Tea tree oil was mixed with the carrier and applied. FIG. 1 shows that application of the carrier did not result in an inflammatory response in the subjects. Further analysis of the presence of myeloperoxidase as a marker for inflammatory cell activity was performed. FIG. 2 shows that tea tree oil with DMSO resulted in the previously observed response, while tea tree oil with the carrier did not. These data confirm that both the carrier and tea tree oil do not trigger inflammation.

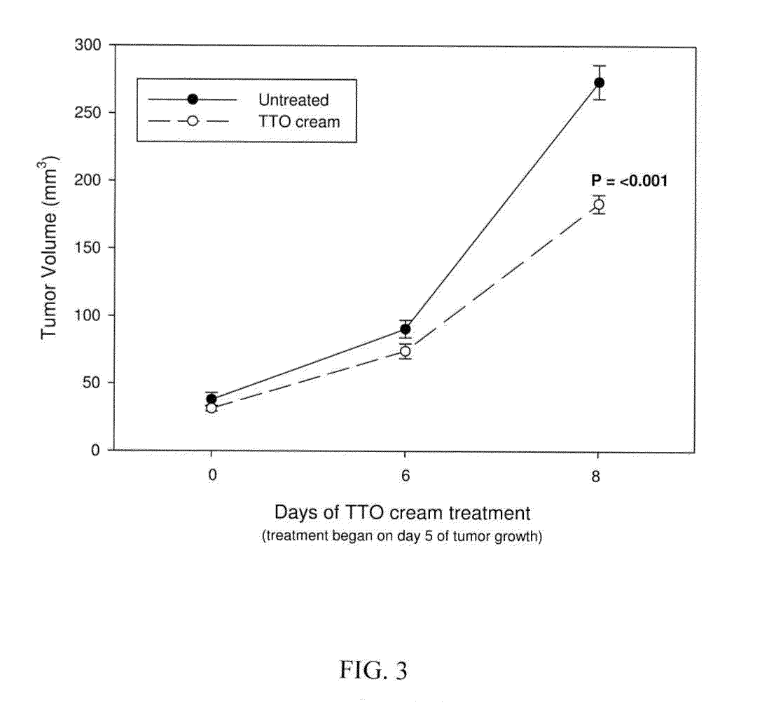

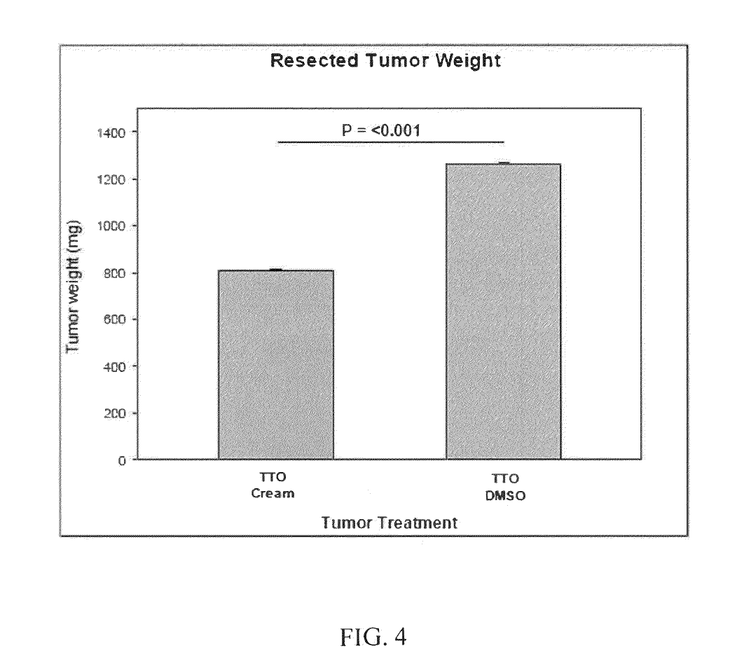

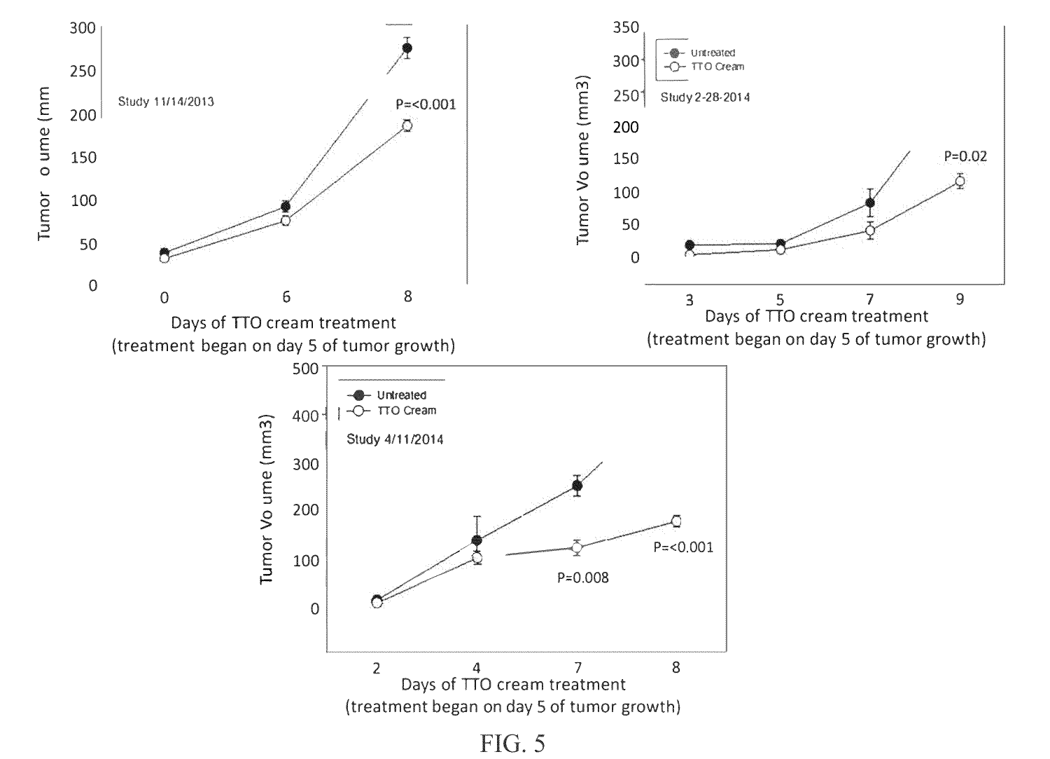

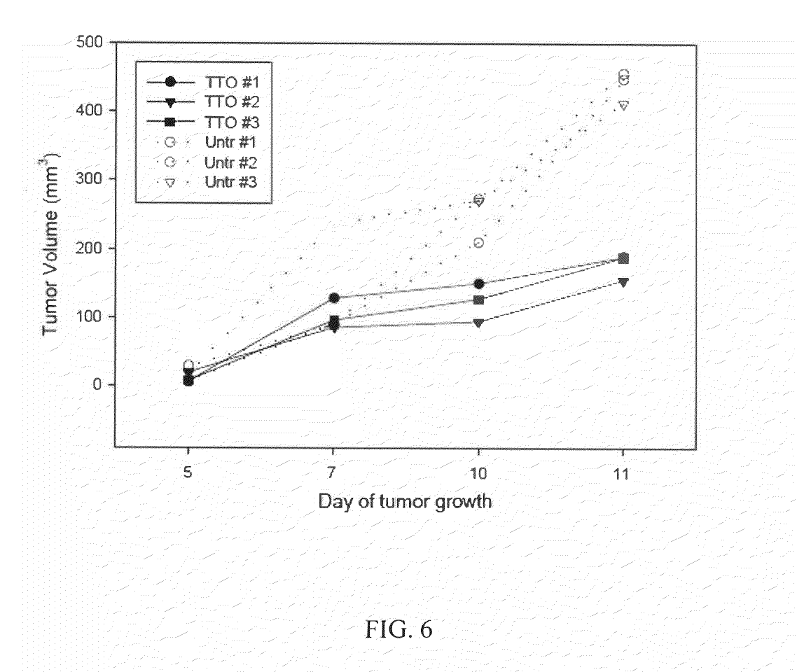

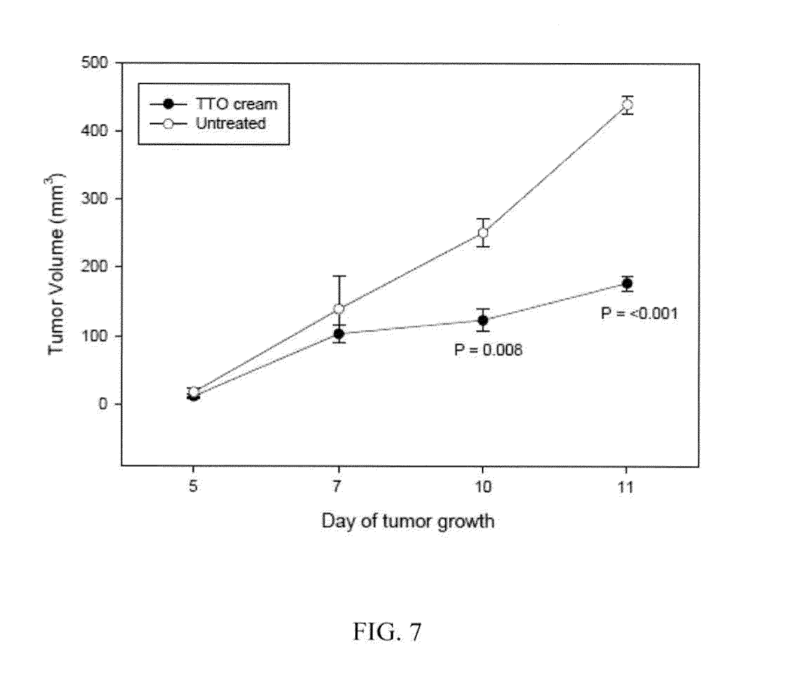

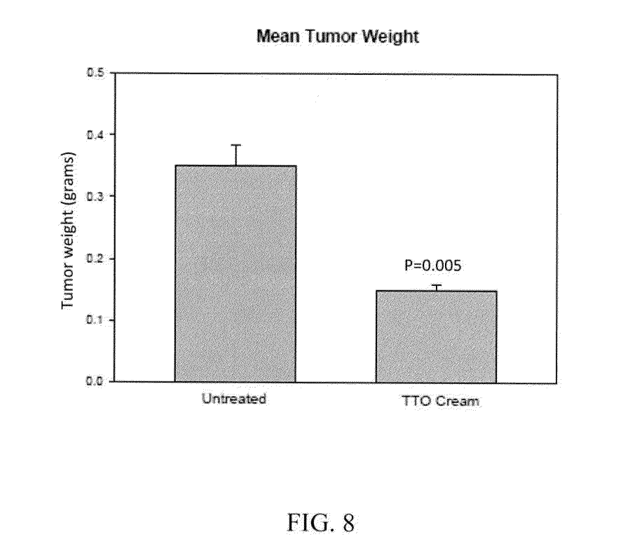

The effect of the TTO with the carrier on the size and weight of the tumors was also assessed. FIGS. 3-8 demonstrate that application of the tea tree oil with the carrier inhibited tumor progression. Collectively, the combination of the tea tree oil and the carrier were effective in delivering the desired therapeutic effect without triggering the undesired inflammatory response.

The foregoing descriptions of various embodiments provide illustration of the inventive concepts. The descriptions are not intended to be exhaustive or to limit the disclosed invention to the precise form disclosed. Modifications or variations are also possible in light of the above teachings. The embodiments described above were chosen to provide the best application to thereby enable one of ordinary skill in the art to utilize the inventions in various embodiments and with various modifications as are suited to the particular use contemplated. All such modifications and variations are within the scope of the invention. All publications, patents and patent applications referenced herein are to be each individually considered to be incorporated by reference in their entirety.

What is claimed:

1. A pharmaceutical composition, comprising:
a therapeutically effective amount of tea tree oil (Melaluca altemifolia); and
a pharmaceutically acceptable carrier.

2. The pharmaceutical composition of claim 1, including between about 9.2 and about 10.8 weight percent tea tree oil and between about 87.95 and about 90.5 weight percent pharmaceutically acceptable carrier.

3. The pharmaceutical composition of claim 1, including about 10 weight percent tea tree oil and about 90 weight percent carrier.

4. The pharmaceutical composition of claim 1, wherein said pharmaceutically acceptable carrier includes water, propylene glycol, phenoxyethanol, heavy paraffin oil, soft white paraffin, cetostearyl alcohol and macrogol cetrostearyl ether.

5. The pharmaceutical composition of claims 1-4 including vanillin.

6. The pharmaceutical composition of claim 5, including between about 0.75 and about 1.25 weight percent vanilla.

7. The pharmaceutical composition of claim 5, including about 1.0 weight percent vanillin.

8. The pharmaceutical composition of claims 4-7, including between about 46.5 and about 49.5 weight percent water, about 4.2 and about 5.8 weight percent propylene glycol, about 0.4 and about 0.8 weight percent phenoxyethanol, about 9.2 and about 10.8 weight percent heavy paraffin oil, about 9.2 and about 10.8 weight percent soft white paraffin, about 14.2 and about 15.6 weight percent cetostearyl alcohol, and about 0.4 and about 1.0 weight percent macrogol cetostearyl ether.

9. The pharmaceutical composition of claims 4-7 including about 47.9 weight percent water, 5.0 weight percent propylene glycol, 0.6 weight percent phenoxyethanol, 10.0 weight percent heavy paraffin oil, 10.0 weight percent soft white paraffin, 14.8 weight percent cetostearyl alcohol and 0.7 weight percent macrogol cetostearyl ether.

10. A method of treating actinic keratosis, comprising topically applying to a subject in need thereof a pharmaceutical composition according to any of claims 1-9.
